(19) United States
(12) Patent Application Publication
(10) Pub. No.: US 2026/0232208 A1
(43) Pub. Date: Aug. 13, 2026

(54) COMPOSITE CATHETER WITH DUAL FUNCTIONS OF ULTRASOUND IMAGING AND FRACTIONAL BLOOD PRESSURE MEASUREMENT

(71) Applicant: THE HONG KONG POLYTECHNIC UNIVERSITY, Hong Kong (CN)

(72) Inventors: Jiyan DAI, Hong Kong (CN); Xuming ZHANG, Hong Kong (CN); Weijin CHEN, Hong Kong (CN)

(21) Appl. No.: 19/442,040

(22) Filed: Jan. 7, 2026

Related U.S. Application Data
(60) Provisional application No. 63/755,365, filed on Feb. 7, 2025.

(51) Int. Cl. A61B 5/0215; A61B 5/00; A61B 5/021; A61M 25/00

(52) U.S. Cl. CPC A61B 5/02154; A61B 5/02141; A61B 5/6852; A61B 5/6876; A61M 25/0043; A61B 2560/0462; A61B 2562/0204; A61B 2562/0219; A61B 2562/0233; A61B 2562/0247; A61B 2562/0266; A61M 2025/0001; A61M 2205/0216; A61M 2205/3303; A61M 2205/3306; A61M 2205/3331; A61M 2205/3375; A61M 2210/12

(57) ABSTRACT

Provided herein is an integrated device with dual functions of ultrasound imaging and optical pressure sensing for intravascular assessment of any potential narrowing or blockage of blood vessels in a subject. The present device integrates both an elastic optical fiber having at least two gratings as pressure sensors with different reflection wavelengths and an ultrasound transducer into a hollow tube-like structure to form an intravascular composite catheter for simultaneous pressure sensing of blood flow flowing through a target blood vessel and imaging of its vessel wall in order to assess the morphology of potential plaques and also the degree of potential stenosis in the target blood vessel. Related methods for fabrication, application of and corresponding diagnostic system incorporating the present device for determining cardiovascular diseases of a subject and evaluating the efficacy of corresponding treatment regime are also provided.

### CROSS-REFERENCE TO RELATED APPLICATIONS

This application claims priority from a U.S. provisional patent application Ser. No. 63/755,365 filed Feb. 7, 2025, the disclosure of which is incorporated herein by reference in its entirety.

### TECHNICAL FIELD

The present invention relates to a device with dual functions of ultrasound imaging and fractional blood pressure measurement for cardiovascular disease diagnosis, in particular, to an intravascular composite catheter incorporating an optical fiber Bragg grating (FBG) and ultrasound composite catheter capable of obtaining vascular wall images via intravascular ultrasound (IVUS) and fractional flow reserve (FFR) data in blood vessels simultaneously.

### BACKGROUND

Coronary artery disease is one of the common cardiovascular diseases (CVD), which was the first cause of death in the world. The coronary arteries are branches of the major arteries that carry blood to the heart muscle with adequate oxygen and nutrients. Most coronary artery diseases are caused by angina pectoris or heart failure caused by insufficient blood supply to the heart due to blockage of the coronary arteries by cholesterol or blood clots, and in severe cases, death. Early detection and cardiovascular dilation surgery are effective in preventing death from coronary artery disease.

Cardiovascular disease detection is based on the standard of Fractional Flow Reserve (FFR). FFR is a test that measures the blood flow ratio at two locations in the coronary artery, based on the theory that the maximum blood flow obtained by the myocardial region supplied by the coronary artery should theoretically be equal to the blood flow in the same region. When there is a stenotic lesion in the vessel, the blood flow ratio decreases; for example, an FFR value of 0.8 indicates that the degree of stenosis in the subject's coronary artery has reduced the maximum blood flow to 80% of normal. When the FFR value is less than 0.6, the subject requires cardiovascular dilation surgery.

Intravascular ultrasound (IVUS) is a medical imaging method used for the auxiliary diagnosis of cardiovascular diseases, employing specially designed catheters with miniature ultrasound probes attached to the distal end. The most valuable application of IVUS is to visualize plaques that are not visible through angiography. It is increasingly utilized in research to better understand the behavior of a patient's atherosclerotic process. IVUS not only allows for accurate observation of the inner walls of coronary arteries but also enables the examination of atherosclerotic plaques that are “hidden” within the vessel walls.

Traditional CVD diagnostic method determines the blood flow ratio by measuring the pressure difference at both ends of the coronary artery using a pressure guide wire made of piezoelectric sensor. The current method of testing intracoronary catheter fractional flow reserve (FFR) is to use a pressure guidewire to test the blood pressure difference at both ends of the blood vessel. However, this conventional detection method can only assess the pressure difference caused by the narrowing or stenosis of a small area of the coronary blood vessel such as coronary artery. It is poor and cannot detect the overall stenosis of blood vessels in a wide area.

There is a need for an improved device or system with both IVUS and FFR functions to be executed simultaneously that eliminates or at least diminishes the disadvantages and drawbacks described above.

### SUMMARY OF INVENTION

In a first aspect, provided herein is a device with dual functions of optical pressure sensing and ultrasound imaging through incorporating both fiber Bragg grating (FBG) and intravascular ultrasound (IVSU) imaging means. The present device comprises an intravascular ultrasound transducer and an optical pressure sensor, the optical pressure sensor comprising an optical fiber having at least two optical fiber Bragg gratings as pressure sensors with different reflection wavelengths and being spaced apart from each other in a first distance; the intravascular ultrasound transducer comprising an ultrasonic imaging probe; the optical pressure sensor and the intravascular ultrasound transducer being sleeved into a hollow tube-like structure forming a bundle of optical fiber and ultrasound transducer.

In certain embodiments, the at least two optical fiber Bragg grating (FBG) sensors are engraved proximal to one end of the optical fiber for measuring a pressure difference between at least two locations of a target site; the other end of the optical fiber is connected to a light source.

In certain embodiments, the target site is located in blood vessels of a human subject.

In certain embodiments, the hollow tube-like structure is a hollow cannula.

In certain embodiments, the cannula has an outer diameter smaller than an inner diameter of the blood vessels; the cannula can be elastic; a front end thereof has an opening; and a circumference thereof is partially open.

In certain embodiments, the blood vessels comprise coronary arteries and the device is an intracoronary catheter.

In certain embodiments, the device is selected from a 3.6 Fr catheter incorporated with a polymer optical fiber having a pair of FBG sensors and a high-frequency ultrasound transducer having an axial resolution of about 50 μm and a resolution in a lateral direction of about 200 μm.

In certain embodiments, the high-frequency ultrasound transducer is made of a ferroelectric single-crystal piezoelectric material.

In certain embodiments, the high-frequency ultrasound transducer is capable of generating ultrasound at a frequency of 50 MHz or above.

In certain embodiments, the polymer optical fiber has a diameter of about 70 μm.

In certain embodiments, the polymer optical fiber is fixedly arranged on an outer wall of a sheath of the ultrasound transducer.

In certain embodiments, the polymer optical fiber is bound onto the outer wall of the sheath of the ultrasound transducer with an adhesive selectively matching the Young's modulus of the polymer optical fiber such that the pair of FBG sensors remain uncompromised which ensures a cured adhesive and the polymer optical fiber undergo strain synchronously under external pressure variations.

In certain embodiments, the cured adhesive has a strength of about 2.4 GPa.

In certain embodiments, the subject is a human.

In a second aspect, provided herein is a detection system comprising the device described herein, an ultrasound imaging system, a fiber Bragg grating (FBG) demodulator, and a user terminal.

In certain embodiments, the light source is selected from a stabilized LED with an emission wavelength from 1450 nm to 1630 nm or a laser light source.

In certain embodiments, the FBG demodulator is selected from a fiber Bragg grating interrogator capable of signal demodulation and data acquisition.

In certain embodiments, the light source connects to one end of the optical fiber of the device while the other end of the optical fiber is provided with the pair of FBG sensors for measuring the pressure difference between two different or multiple locations of the target site.

In certain embodiments, the detection system further comprises a photodiode and an optical gyroscope fiber circulator disposed between the light source and the optical fiber for receiving and transmitting reflected optical signal from the optical fiber.

In a third aspect, provided herein is a method of diagnosing cardiovascular diseases comprising applying the device or system described herein to a subject.

In certain embodiments, the method further comprises inserting one end of the device into a blood vessel of the subject for obtaining ultrasound images and measuring a first pressure value and a second pressure value at two different locations of a target site simultaneously, calculating a pressure difference between the first pressure value and the second pressure value, thereby determining fractional flow reserve in the blood vessel.

In certain embodiments, the device is moved to the target site where the blood vessel is susceptible to be narrowed or blocked which affects normal function of the subject's heart or cardiovascular system.

In certain embodiments, the other end of the device is connected to the light source for providing a light emission to the optical fiber of the device.

In certain embodiments, the light emission from the light source is reflected by the two optical fiber Bragg gratings to form two monochromatic optical signals with different wavelengths.

In certain embodiments, the reflected optical signals from the corresponding optical fiber Bragg gratings of the optical fiber are transmitted to a photodiode through an optical gyroscope fiber circulator disposed at and connected between the light source and the optical fiber for determining the corresponding pressure values, thereby calculating the pressure difference between the corresponding pressure values in order to determine the fractional flow reserve of the blood vessel.

In certain embodiments, the subject is human and the blood vessel is coronary artery.

In certain embodiments, the first distance between the two optical fiber Bragg gratings is at least 2 cm.

In certain embodiments, each of the two optical fiber Bragg grating sensors has a corresponding wavelength shift in response to surrounding pressure change under uniform temperature changes in accordance with the following formulae:

$$Δ {λ}_{FBG 1} = {K}_{ε} {ε}_{1} ; \qquad ( 3 )$$

$${Δλ}_{FBG 2} = {K}_{ε} {ε}_{2} , \qquad ( 4 )$$

wherein K_{ε} is strain coefficient related to an elastic-optical effect of the corresponding fiber Bragg grating; ε₁ or ε₂ is an individual strain measured by the corresponding fiber Bragg grating.

In certain embodiments, the pressure difference between the first and second pressure values determined from the strain measured by the corresponding fiber Bragg grating is determined by the following formula:

$$ε = \frac{Δ {λ}_{FBG 1} - Δ {λ}_{FBG 2}}{{K}_{ε}} ; \qquad ( 5 )$$

and the stress monitored by the optical fiber of the device can be approximated by the following formula under Hooke's law:

$$σ = E \cdot ε , \qquad ( 6 )$$

wherein E denotes an elastic modulus of the optical fiber.

In certain embodiments, the optical fiber is selected from a polymer optical fiber with the elastic modulus of approximately 2.4 GPa.

In certain embodiments, the device has a detection limit for blood pressure variations as low as 160 Pa or about 1% of a human standard physiological blood pressure.

In certain embodiments, the cardiovascular diseases comprise coronary artery diseases such as coronary atherosclerosis, ischemia, myocardial infarction, stroke, and aortic stenosis, or any diseases, symptoms or conditions associated with stenosis.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 is a block diagram depicting a detection system of the present invention according to certain embodiments;

FIG. 2A schematically depicts from a side view a device of the present invention according to certain embodiments;

FIG. 2B schematically depicts from a cross-sectional view of a device of the present invention according to certain embodiments;

FIG. 3 schematically depicts the present FBG sensors arranged with the present ultrasound transducer to form the composite catheter according to certain embodiments of the present invention;

FIG. 4A shows a schematic of polymer optical fiber Bragg grating (POFBG) strain measurement and pressure sensitivity calibration curves of the dual function POFBG;

FIG. 4B shows initial spectra of the dual FBG embedded in the polymer optical fiber;

FIG. 4C shows calibration curves for pressure sensitivity of the dual function POFBG;

FIG. 5A an image of an ultrasound standard phantom captured by the present device in axial direction;

FIG. 5B shows an image of the ultrasound standard phantom captured by the present device in lateral direction;

FIG. 5C shows an image of the ultrasound standard phantom captured by the present device in a direction towards the depth of 3 mm of the phantom;

FIG. 5D shows a single-frame image captured by the present device at 0.1 mm position during 1-second pullback;

FIG. 5E shows a merged image of 1004-frame images captured by the present device at 100 mm position during 10-second pullback;

FIG. 6A shows a schematic of localized stenotic structure in a simulated vessel;

FIG. 6B shows measured wavelength shifts for a first fiber Bragg grating (FBG 1) and a second fiber Bragg grating (FBG 2) by the present device in a vascular simulation apparatus, where the normalized FFR variation is based on the wavelength shift;

FIG. 6C shows the wavelength shift process of FBG1 and FBG2 when the stenosis moves between 0~6 mm;

FIG. 6D shows the change in pressure difference detected/determined by the present device/system when the stenosis moves between 0~6 mm;

FIG. 7A shows a fluoroscopic image by DSA fluoroscopy of coronary artery during interventional procedure in a large-animal porcine model captured by the present device;

FIG. 7B shows an image of the porcine coronary vasculature captured by the present device;

FIG. 7C shows Intravascular pressure variation in coronary arteries during porcine intervention;

FIG. 7D shows the normalized FFR variation in coronary arteries during porcine intervention;

FIG. 8 depicts a fabrication method of the optical fiber according to certain embodiments;

FIG. 9A shows Tg measurement of the fabricated optical fiber prepared according to the embodiments depicted in FIG. 8 and Example 5;

FIG. 9B shows an expanded view of a selected segment of FIG. 9A indicating a measured Tg of 137.7° C.;

FIG. 10A shows a plot of stress-strain curve for the fabricated optical fiber with a diameter of 160 μm and a length of 1 cm prepared according to the embodiments depicted in FIG. 8 and Example 5;

FIG. 10B shows Young's modulus measurements of the fabricated optical fiber with a diameter of 160 μm and a length of 1 cm prepared according to the embodiments depicted in FIG. 8 and Example 5.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides a device incorporating an elastic optical fiber Bragg grating (FBG) and ultrasonic composite catheter that can effectively address the drawbacks in the existing technologies. Since the optical fiber Bragg grating is extremely sensitive to pressure feedback, it provides stronger resolution and more accurate intravascular pressure measurements than traditional catheter pressure guide wires. Utilizing optical fiber Bragg technology allows for two different blood pressure feedbacks to be achieved within the same catheter. The ultrasonic imaging catheter of the present invention detects narrowing or blockage of blood vessels, e.g., stenosis, while the fiber Bragg grating pressure sensor measures the blood flow reserve fraction before and after the stenosis. The data from both are compiled at the data terminal and analyzed intelligently by the computer, allowing the physician to clearly understand the condition of the plaque, providing critical criteria for the use of stents. This integrated design also offers a method for physicians to assess the treatment effects on patients with cardiovascular diseases.

Certain embodiments of the present invention provide an integrated device composite ultrasound imaging and fiber Bragg grating guiding tubes to simultaneously obtain vascular wall imaging and the fractional flow reserve (FFR) of blood flow in the vessels, aiming to address the shortcomings of traditional pressure guidewire methods in diagnosing cardiovascular diseases. By employing a composite catheter for a single detection in the present invention, a real-time synchronous acquisition of both FFR and IVUS data for cardiovascular disease (CVD) patients is achieved, along with real-time intelligent analysis by the detection system, facilitating an efficient, accurate, and intelligent diagnosis of CVD.

The wavelength of the reflected light wave of the fiber Bragg grating is determined by the formula λ_{B}=2nₑΛ, where Λ is the spacing between the stripes and nₑ is the effective refractive index of the fiber core. The bandwidth of the light waves reflected by fiber Bragg gratings is very narrow, and the attenuation of the optical signal during transmission in the fiber is minimal, making it easy for instruments to distinguish the received signals. Compared to traditional technologies, the light waves reflected by fiber Bragg gratings are highly selective, allowing for the presence of more than one Bragg grating within a single fiber in applications, enabling a single fiber to obtain multiple pressure feedback points simultaneously within a blood vessel. Consequently, the diameter of the catheter can be reduced, resulting in smaller wounds for the test subjects and less discomfort during the testing process. According to the formula described herein, the fiber Bragg gratings exhibit a significant wavelength shift for very small changes in length, making the fiber extremely sensitive to pressure changes, which is suitable for measuring fluid pressure values such as blood pressure.

One important feature of the present invention is that the catheter is equipped with an ultrasonic imaging catheter for real-time imaging of the coronary artery. Traditional pressure guidewires used for FFR detection cannot identify the issue of insufficient blood flow caused by overall average blockage in the coronary artery, as the artery with overall average blockage will experience a significant drop in blood flow over a long distance. If this distance exceeds the distance between the two sensors of the pressure guidewire, the pressure difference measured at both ends of the guidewire will be minimal, resulting in a calculated FFR that conforms to standard values. By utilizing ultrasonic imaging technology in the present invention, images of the cross-section of the artery can be obtained, clearly displaying whether there is any blockage or narrowing at each position within the artery. This allows the catheter in the present invention to perform two tests simultaneously, enabling a more effective, time-saving, and accurate assessment of whether a patient requires surgery on the coronary artery.

Although the following examples take coronary artery as a target site for disease diagnosis by the present device or system for illustration purposes, they should not be considered limiting the application of the present invention to only coronary artery.

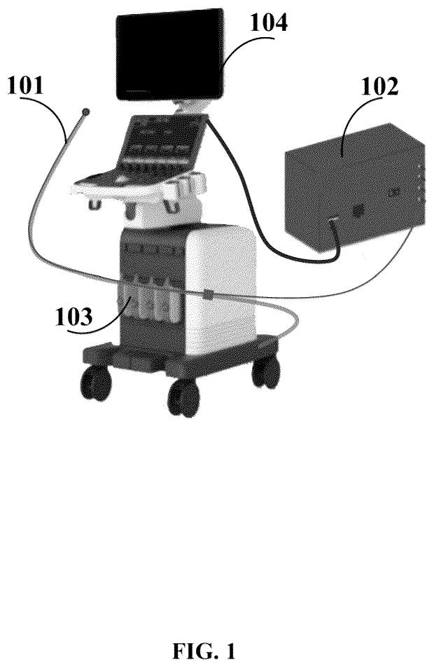

Turning into FIG. 1, a detection system 1 according to an embodiment is depicted, comprising an integrated device 101 of the elastic optical fiber Bragg grating and ultrasound transducer for disease diagnosis to obtain vascular wall images via intravascular ultrasound (IVUS) and detect fractional flow reserve (FFR) data in blood vessels simultaneously. The detected data is collected through the optical fiber grating demodulation system 102 and the ultrasound imaging system 103, and the collected data is summarized at the data terminal 104 for processing. It provides real-time online feedback on the condition of the subjects through programmatic intelligent analysis for clinical diagnostic reference. The diagnosis broadly covers the diseases associated with stenosis including, but not limited to, coronary artery diseases, coronary atherosclerosis, ischemia, myocardial infarction, stroke, aortic stenosis, etc., or any other cardiovascular diseases related to or associated with stenosis.

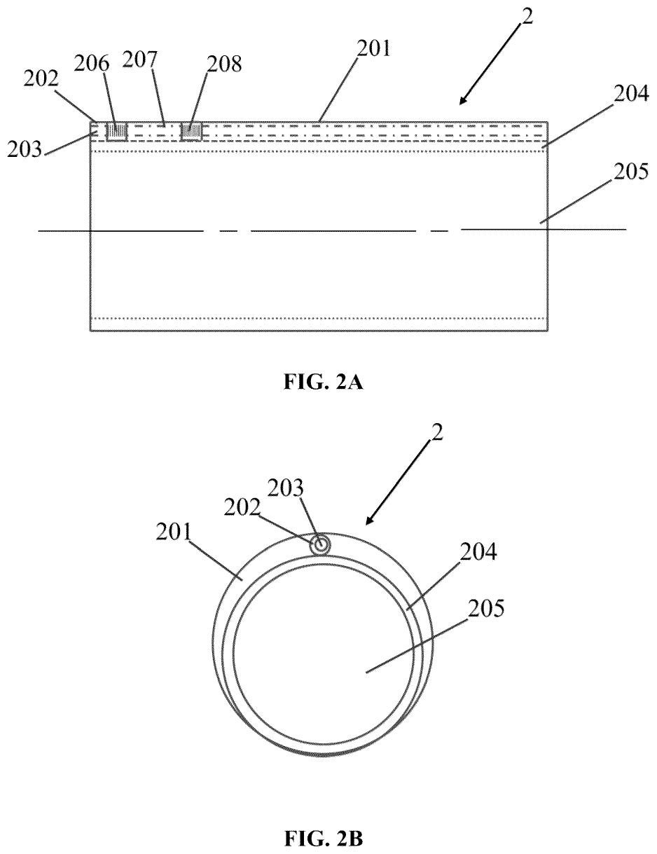

Turning into FIG. 2A, the integrated device 2 according to an embodiment is depicted, comprising a hollow sleeve 201 with an outer diameter smaller than that of the coronary vessels and having elasticity, an optical fiber 202 engraved with at least two fiber Bragg gratings (FBG) (206, 208) being pressure sensors, and an ultrasound catheter 204. The hollow sleeve 201 has hole-like structures at the two grating areas 206 and 208 of the optical fiber 202; the optical fiber 202 has gratings (206, 208) with different reflection wavelengths etched within the core 203, and the two Bragg gratings (206, 208) are spaced a certain distance 207 apart, with no specific limitations on the distance 207; the ultrasonic catheter 204 contains an ultrasonic imaging probe 205. FIG. 2B schematically depicts the arrangement of different components in the integrated device 2 from its cross-sectional view.

In certain embodiments, high-frequency (50 MHz) ultrasound transducer is used for performing ultrasonic imaging. The ultrasound transducer can be made of ferroelectric single-crystal piezoelectric material or any piezoelectric materials that can be used for ultrasound transducer.

In certain embodiments, polymer optical fiber (POF) is selected as the optical fiber as it provides certain elasticity and can be etched to create fiber Bragg gratings (FBG) using conventional methods such as UV phase mask technique.

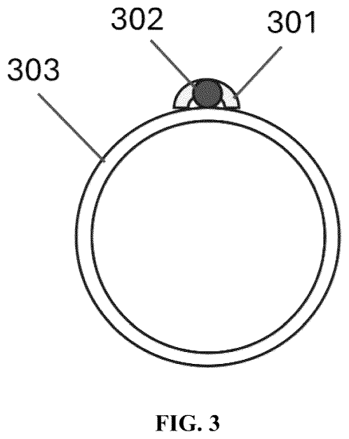

To match the Young's modulus of the selected optical fiber, a modified acrylate adhesive is employed for bonding the FBG sensor onto a sheath of the ultrasonic transducer. Other adhesive that can match the corresponding modulus of the selected optical fiber can be used in the present invention. In certain embodiments, the adhesive for said bonding after curing achieves an approximate strength of 2.4 GPa, matching the Young's modulus of the POF. This modulus matching ensures the cured adhesive and the POF undergo strain synchronously under external pressure variations. As a result, the POFBG sensor formed as such can accurately detect the pressure changes in the target site of disease diagnosis. FIG. 3 depicts an embodiment that two FBG sensors 301 are fixedly arranged on a sheath of ultrasound transducer 302 with epoxy 303 to form the composite catheter of the present invention.

Fabrication of certain embodiments of the present POF is illustrated in the following Examples 5 and 6.

### EXAMPLES

### Example 1—Pressure Gradient Assessment and Calibration of FBG Pressure Sensors of Present Device

An initial pressure gradient assessment across simulated coronary stenoses is conducted using polydimethylsiloxane (PDMS) vascular phantoms. The present device (or “composite catheter” used interchangeably herein) is inserted into the phantom vessel, with the optical fiber sensing region securely positioned at a specific location to ensure stability during induced intravascular pressure variations. Pressure sensitivity characterization is performed by systematically modulating the applied pressure within the physiological human vascular pressure range (0-15 kPa) using a pressure control unit. The corresponding wavelength shift of the fiber Bragg grating pressure sensor is measured under each pressure condition. A fiber Bragg grating interrogator is employed for signal demodulation and data acquisition. During interrogator operation, broadband light from a stabilized LED source (emission wavelength range: 1450-1630 nm) is coupled into the FBG sensor, and the FBG-reflected light is directed back to the interrogator. Dynamic pressure gradient changes across the simulated stenosis are determined by monitoring the wavelength shifts of the dual FBG reflection peaks recorded by the interrogator.

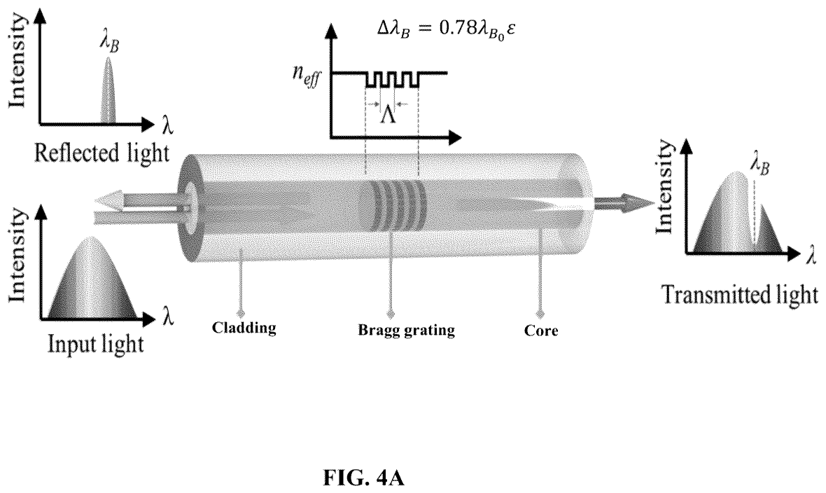

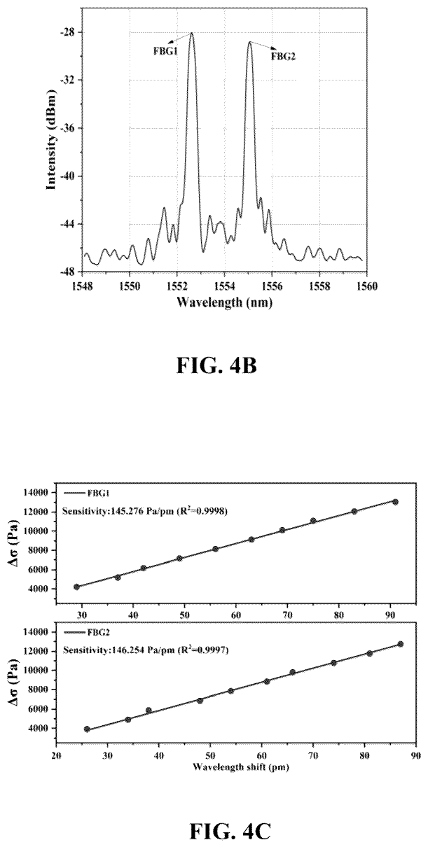

As seen from FIG. 4A, the applied FBG being a stress sensor is an optical fiber whose refractive index is modulated periodically along the core, wherein the grating has a reflection/transmission effect on light in a fixed wavelength range, and wherein the Bragg wavelength of the reflected light satisfies the following relation:

$${λ}_{B} = 2 {n}_{eff} Λ \qquad ( 1 )$$

where n_{eff} is the effective refractive index of the core and Λ is the grating period of the FBG.

When the stress (F) or temperature (T) in the external environment of FBG sensor changes, it will lead to the change of n_{eff} or Λ, which will lead to the shift of Bragg wavelength λ_{B} of reflected light. Therefore, the mathematical relationship between the shift of Bragg wavelength of FBG sensor reflection spectrum and external environmental stress and temperature can be simply expressed by the following formula:

$$Δ {λ}_{B} = {K}_{T} Δ T + {K}_{ε} ε \qquad ( 2 )$$

where K_{T} is the temperature coefficient related to the thermal expansion effect and photothermal effect of FBG, and K_{ε} is the strain coefficient related to the elastic-optical effect of FBG. Since the stress is usually expressed by microstrain in FBG sensors, the stress is replaced by strain ε here.

As observed from the above formula (2), the present FBGs exhibit sensitivity to both strain in the surrounding environment and temperature variations. However, the present fiber-optic pressure sensor employs two FBGs, so under uniform temperature changes, both FBGs demonstrate identical thermal responses. FIG. 4B illustrates the reflection spectra of this dual-FBG pressure sensor, where the two gratings are separated by a minimal distance of 2 cm. With this setup, temperature-induced perturbations are eliminated when calculating the pressure differential through signal subtraction. The wavelength shifts of FBG1 and FBG2 can be described as:

$$Δ {λ}_{FBG 1} = {K}_{ε} {ε}_{1} \qquad ( 3 )$$

$${Δλ}_{FBG 2} = {K}_{ε} {ε}_{2} \qquad ( 4 )$$

By subtracting Formula (3) from Formula (4), the strain measured by the intravascular optical fiber can be expressed as:

$$G = \frac{Δ {λ}_{FBG 1} - Δ {λ}_{FBG 2}}{{K}_{ε}} \qquad ( 5 )$$

Within a defined stress range, an optical fiber can be considered as an ideal elastic body. Thus, within the elastic regime, the stress monitored by FBG sensors can be approximated using Hooke's law, as shown in Formula (6):

$$σ = E \cdot ε \qquad ( 6 )$$

where E denotes the elastic modulus of the optical fiber, e.g., the ZEONEX based polymer optical fiber (POF) employed in this example exhibits an elastic modulus of approximately 2.4 GPa.

Calibration tests for the pressure of the composite catheter are performed within the human blood pressure range, i.e., 0~15 kPa. As seen from FIG. 4C, the wavelength shift of the POFBG sensor under varying pressure conditions demonstrates a composite catheter pressure sensitivity of 6.81 μm/kPa, which reveals that the present POFBG sensor exhibits definitive detection capability for blood pressure variations as small as 160 Pa (or equivalent to 1% of the standard human physiological pressure which is about 16 kPa).

### Example 2—Imaging Performance Assessment of Present Device

To validate the superior imaging performance of the ultrasound transducer of the composite catheter, an ultrasound standard phantom is employed to systematically examine the resolution, imaging depth, and frame rate of the present ultrasound transducer. Additionally, the center frequency and bandwidth of the ultrasound transducer are measured using the pulse-echo method. Upon confirming that all technical specifications of the composite catheter meet the application requirements, in vivo coronary intervention procedures are then performed on adult swine. During the surgical procedures, spectral wavelength variations and ultrasound images were synchronously acquired using a 100 Hz fiber Bragg grating interrogator alongside the ultrasound imaging system.

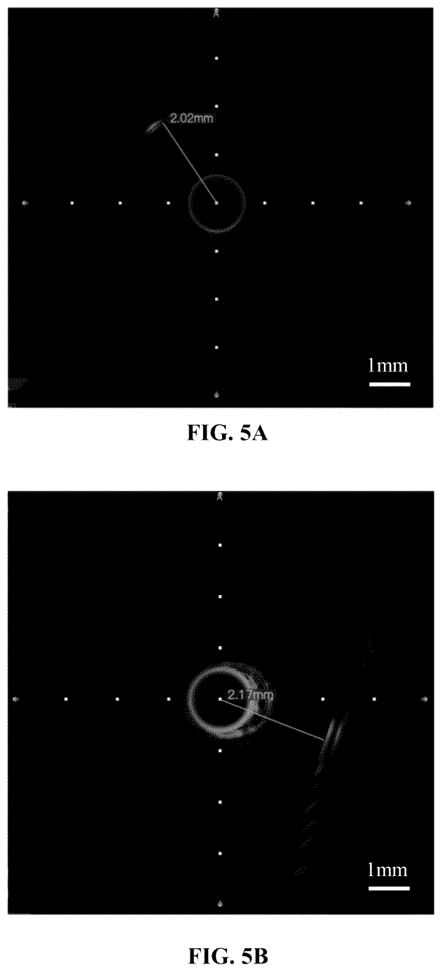

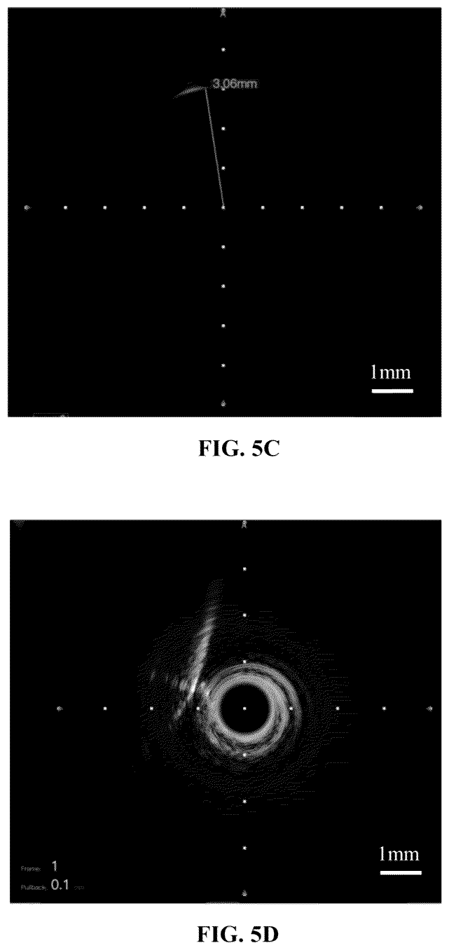

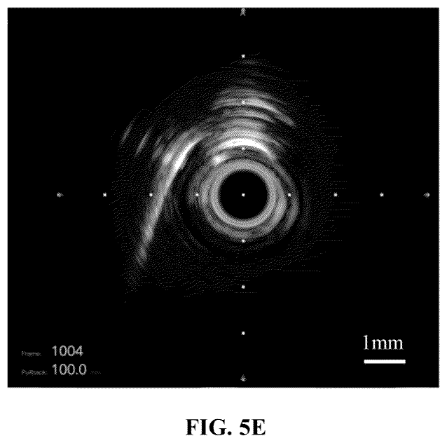

As seen from FIGS. 5A-5E, the images demonstrate that at approximately 2 mm from the working catheter tip, the composite catheter could clearly resolve targets separated by 50 μm in the axial direction and 200 μm in the lateral direction. Additionally, targets located at a depth of 3 mm are clearly visualized. During a 10-second pullback over 100 mm, 1004 image frames are acquired, yielding an imaging frame rate of 100 frames/s and a pullback speed of 10 mm/s. These transducer specifications align with clinically applicable IVUS imaging performance requirements, indicating that the integration of the present POFBG sensor does not compromise the imaging performance of the ultrasound catheter.

### Example 3—In Vitro Simulated Stenotic Lesion Testing for Present Device

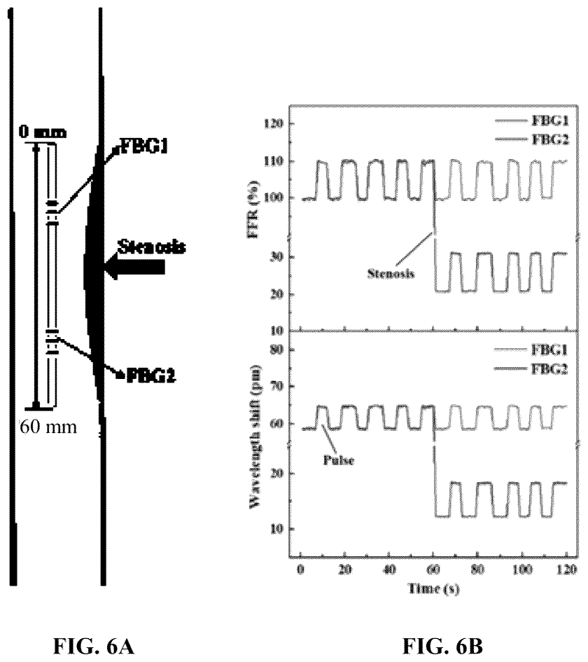

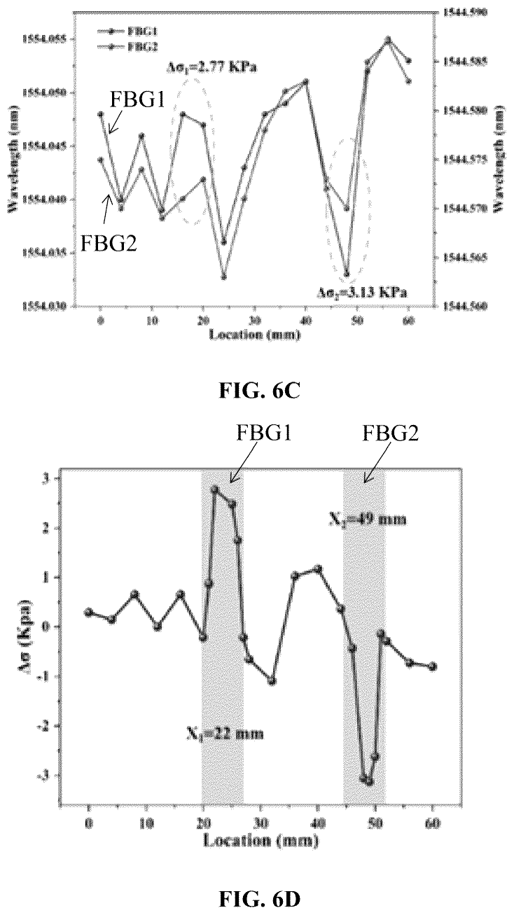

A hemodynamic simulation system (not shown in Figures) is employed to position the IVUS-FFR composite catheter within a simulated vessel. Pressure modulation is achieved via pressurized infusion bags, while wavelength shifts of the two FBG sensors of the present device are recorded using an interrogator, enabling quantification of pressure differentials across simulated stenotic lesions. The degree of stenosis is controlled by a medical infusion flow regulator, an example of which is shown in FIG. 6A that is a schematic of the stimulated stenosis and location of the dual-FBG. In order to determine the maximum pressure difference that can be detected at narrow points by the optical fiber, a fixed position of the optical fiber is maintained during testing while adjusting the position of the control valve, ensuring consistent narrowness within the range of 0 to 6 mm (FIG. 6A).

As seen from FIGS. 6C-6D, the detected pressure difference is maximized when the narrow point is near a certain FBG grating point, with the highest detected pressure difference of approximately 3.13 KPa occurring when the narrow position is close to FBG2. Therefore, the pressure difference detected when the narrow position is near FBG2 is used as the pressure difference between the narrow position and the normal position in the rest of the examples. Based on pressure differentials, FFR variations are plotted (FIG. 6B). Notably, under normal vascular conditions, wavelengths exhibit pulsatile fluctuations synchronized with pressurization frequency, with consistent shifts observed in both FBGs; without stenosis, the corresponding FFR value remains at 100%. However, when the catheter is advanced to the stenosis site, FBG2 exhibits a significant central wavelength shift relative to FBG1, indicating detectable pressure gradients proximal and distal to the stenosis. It suggests that the present composite catheter allows clinicians to efficiently capture FFR data near pathological sites in a single procedure.

### Example 4—In Vivo Interventional Study of Present Device in a Porcine Model

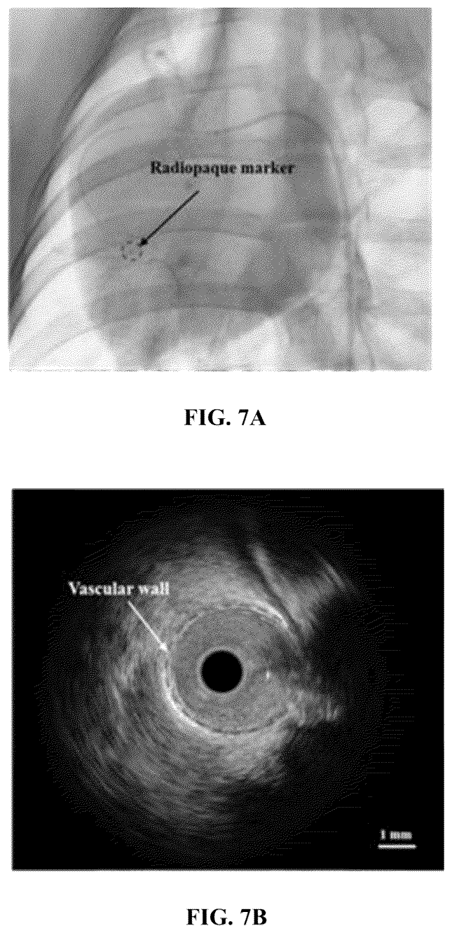

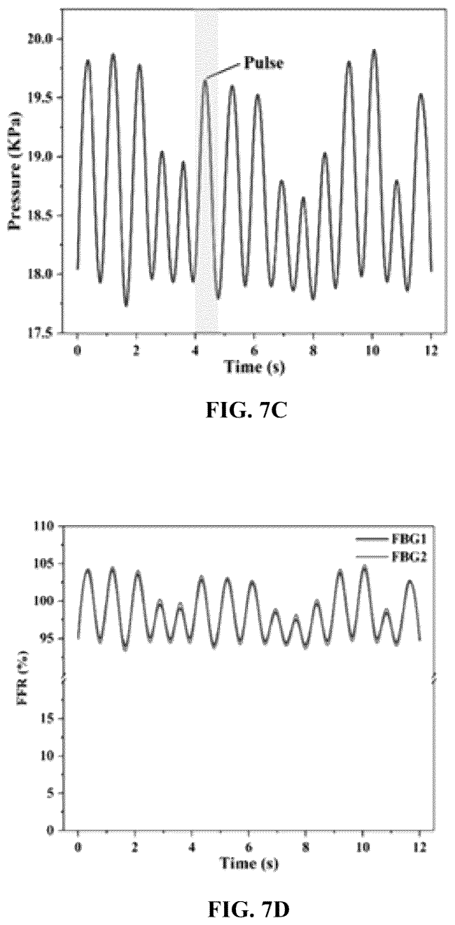

Following successful phantom testing and ex vivo tissue evaluations demonstrating satisfactory image quality in the foregoing examples, in vivo coronary angiography and vascular imaging studies in porcine models are conducted for the present composite catheter. During the procedure, a 0.072-inch guiding catheter is positioned at the coronary ostium, followed by advancement of a 0.014-inch guidewire to the distal segment of the target vessel. The composite catheter is then delivered over the guidewire to the distal region of interest. Fluoroscopic image (FIG. 7A) confirms appropriate positioning within the porcine coronary artery. The integrated ultrasound system's rotational motor is subsequently activated, enabling 360° real-time imaging via the distal transducer. Continuous pullback from distal to proximal segments is performed at a controlled speed, and synchronous acquisition of ultrasonic imaging data and spectral measurements is maintained throughout pullback. FIG. 7B depicts an ultrasonic imaging of the porcine coronary artery confirming normal coronary vasculature morphology, while FIG. 7C demonstrates physiological intracoronary pressure waveforms synchronized with cardiac cycles. Analysis of these pressure fluctuations indicates systolic pressure and diastolic pressure of around 17.5-20 KPa with a heart rate of 75 beats per minute. It should be noted that since the porcine model does not have stenosis, the FFR value measured from the two FBGs is constant 100%, as shown in FIG. 7D. It is reasonably foreseeable to show FFR results with a value of less than 100% in clinical trials on patients with cardiac disease by using the present invention.

### Materials and Methods

### Example 5—Fabrication of ZEONEX SMPOFs

The ZEONEX-based all-solid-single-mode step-index POFs (SIPOFs) used in certain embodiments of the present invention were fabricated using the pull-through method. The ZEONEX SMPOF composed of a single monomer material, was fabricated using different grades of ZEONEX material, where the core (E48R) has a slightly higher refractive index than that of the cladding (480R). The glass transition temperatures (Tg) of the two grades of ZEONEX are ~138° C. which denotes that the fiber can operate at a higher temperature than that of PMMA.

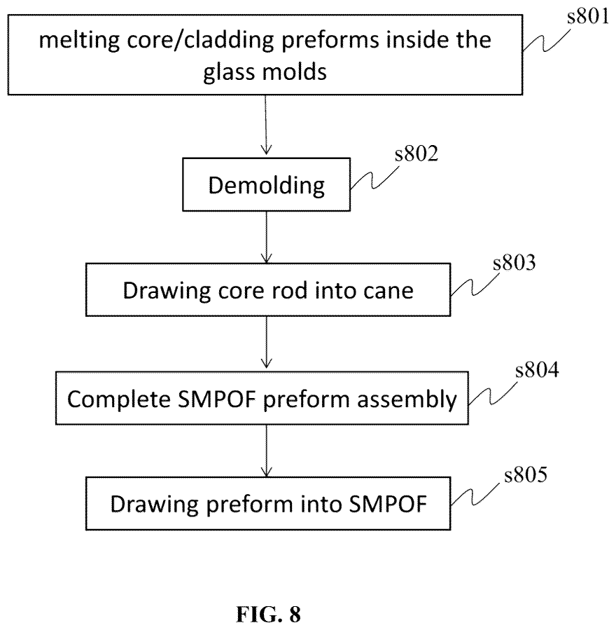

FIG. 8 illustrates the fabrication process of pull through method used in this example, in which the core material, ZEONEX E48R, was melted inside a glass tube and the cladding material, ZEONEX 480R, was melted inside another glass tube where a Teflon wire with a diameter of 0.9 mm was placed at the center (s801). Afterwards, the Teflon wire was removed to form a hole in the cladding preform (s802). The entire melting process was conducted inside a vacuum oven under a maximum temperature of 245° C. to remove O₂ and air. The core material was then drawn into a cane (s803), with a diameter similar to that of the Teflon wire. The core cane was then inserted inside the cladding to form the complete polymer preform assembly (s804), which was eventually drawn into SMPOF (s805) with a diameter of 70 μm.

### Example 6—Physical Properties of SMPOFs

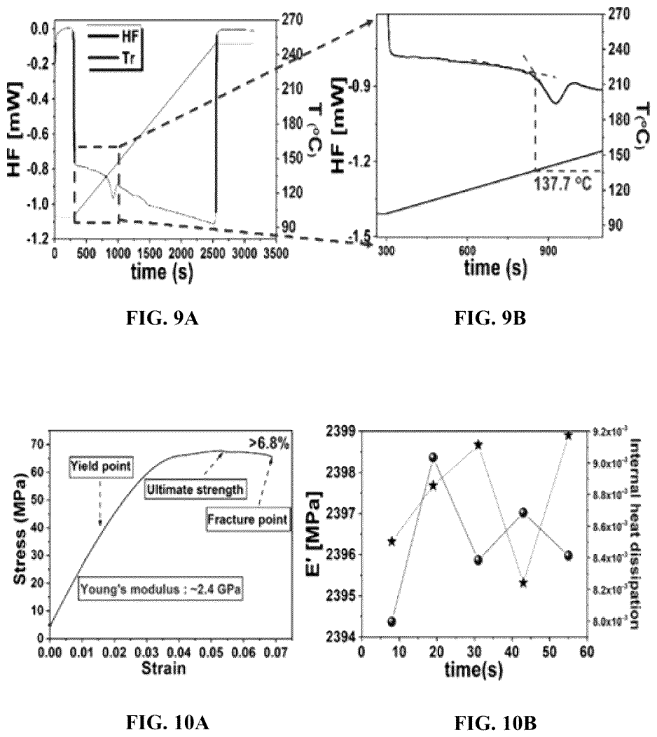

The SMPOFs prepared according to Example 5 were tested by measuring Tg and stress-strain thereof. Tg of the fabricated SMPOFs was measured using a differential scanning calorimetry (DSC) machine (METTLER TOLEDO, Model: DSC3). The heat absorption characteristics of an SMPOF sample was analyzed by heating the sample at a ramping rate of 4° C./min over a temperature range from 100° C. to 250° C. under a constant nitrogen gas flow of 50 ml/min. FIGS. 9A-9B show the heat flow of the SMPOF sample during the heating process in the DSC machine. The measured value of Tg is 137.7° C. which is as same as that of ZEONEX 480R specified by the manufacturer. This verifies the absence of any chemical reaction during the SMPOF fabrication process. Stress-strain measurements based on dynamic mechanical analysis (DMA) method were performed to characterize the mechanical properties of the SMPOFs. The investigation was carried out using a DMA machine (METTLERTOLEDO, Model: DMA1) on a 70 μm diameter SMPOF sample.

FIG. 10A shows the measured tensile strength of the SMPOF sample and FIG. 10B depicts its Young's modulus measurements which are derived from the linear region of the stress-strain curve in FIG. 10A. The result shows that the average Young's modulus is ~2.4 GPa and the elongation-at-break is over 6.8% elongation. From FIG. 10B, the measured value of internal dissipation, which is ~8×10⁻³ indicates that the energy loss at each measurement point is considerably small. This denotes that, when the SMPOF is subjected to an elongation, the energy transferred from mechanical energy to thermal energy is rather less and hence, results in only a slight temperature change during the Young's modulus measurement.

Although the invention has been described in terms of certain embodiments, other embodiments apparent to those of ordinary skill in the art are also within the scope of this invention. Accordingly, the scope of the invention is intended to be defined only by the claims which follow.

What is claimed is:

1. A device with dual functions of optical pressure sensing and ultrasound imaging comprising an intravascular ultrasound transducer and an optical pressure sensor, the optical pressure sensor comprising an optical fiber having at least two optical fiber Bragg grating (FBG) sensors with different reflection wavelengths and being spaced apart from each other in a first distance; the intravascular ultrasound transducer comprising an ultrasonic imaging probe; the optical pressure sensor and the intravascular ultrasound transducer being sleeved into a hollow tube-like structure.

2. The device of claim 1, wherein the at least two optical FBG sensors are engraved proximal to one end of the optical fiber for measuring a pressure difference between at least two locations of a target site; the other end of the optical fiber is connected to a light source.

3. The device of claim 1, wherein the target site is located in blood vessels of a subject.

4. The device of claim 3, wherein the hollow tube-like structure is a hollow cannula.

5. The device of claim 4, wherein the cannula has an outer diameter smaller than an inner diameter of the blood vessels; the cannula is elastic; a front end of the cannula has an opening; and a circumference thereof is partially open.

6. The device of claim 3, wherein the blood vessels comprise coronary arteries and the device is an intracoronary catheter.

7. The device of claim 1, wherein the device is selected from a 3.6 Fr catheter incorporated with a polymer optical fiber having a pair of the optical FBG sensors and a high-frequency ultrasound transducer having an axial resolution of about 50 μm and a resolution in a lateral direction of about 200 μm.

8. The device of claim 7, wherein the high-frequency ultrasound transducer is made of a ferroelectric single-crystal piezoelectric material.

9. The device of claim 7, wherein the high-frequency ultrasound transducer is capable of generating ultrasound at a frequency of 50 MHz or above.

10. The device of claim 7, wherein the polymer optical fiber has a diameter of about 70 μm.

11. The device of claim 7, wherein the polymer optical fiber is fixedly arranged on an outer wall of a sheath of the ultrasound transducer.

12. The device of claim 7, wherein the polymer optical fiber is bound onto the outer wall of the sheath of the ultrasound transducer with an adhesive selectively matching the Young's modulus of the polymer optical fiber such that the pair of the FBG sensors remain uncompromised which ensures a cured adhesive and the polymer optical fiber undergo strain synchronously under external pressure variations.

13. The device of claim 12, wherein the cured adhesive has a strength of about 2.4 GPa.

14. The device of claim 3, wherein the subject is a human.

15. A detection system comprising the device of claim 1, an ultrasound imaging system, a fiber Bragg grating (FBG) demodulator, and a user terminal.

16. The detection system of claim 15, wherein the light source is selected from a stabilized LED with an emission wavelength from 1450 nm to 1630 nm or a laser light source.

17. The detection system of claim 15, wherein the FBG demodulator is selected from a fiber Bragg grating interrogator capable of signal demodulation and data acquisition.

18. The detection system of claim 15, wherein the light source connects to one end of the optical fiber of the device while the other end of the optical fiber is provided with the pair of FBG sensors for measuring the pressure difference between at least two different locations of the target site.

19. The detection system of claim 15, further comprising a photodiode and an optical gyroscope fiber circulator disposed between the light source and the optical fiber for receiving and transmitting reflected optical signal from the optical fiber.

20. A method of diagnosing cardiovascular diseases comprising applying the detection system of claim 15 to a subject.
